(19) United States
(12) Patent Application Publication
(10) Pub. No.: US 2024/0216014 A1
(43) Pub. Date: Jul. 4, 2024

(54) Flexible Surgical Halo

(71) Applicant: Quinton Brady Brown, Spokane, WA (US)

(72) Inventor: Quinton Brady Brown, Spokane, WA (US)

(21) Appl. No.: 18/308,660

(22) Filed: Apr. 27, 2023

Related U.S. Application Data
(60) Provisional application No. 63/335,790, filed on Apr. 28, 2022.

(51) Int. Cl. A61B 17/34; A61B 17/00; A61B 90/00

(52) U.S. Cl. CPC A61B 17/3421; A61B 17/00234; A61B 90/39; A61B 2017/3425; A61B 2090/3966

(57) ABSTRACT

Tools and techniques for a flexible surgical halo are described. Flexible surgical halo tools and techniques are usable to impede a flow of a liquid and route the liquid away from a device attached to or beneath the flexible surgical halo. In one example, a flexible surgical halo includes an elastically deformable body including a tube portion and a canopy portion. The tube portion is configured for attachment to an outer surface of a device, and the canopy portion is configured to route liquids along an upper surface or an outer edge of the canopy. The canopy portion has a lower surface configured to ensure that liquid is unable to reach the tube portion. In this way, flexible surgical halo tools and techniques provide the ability to shield a surgical instrument from becoming soiled by liquid inside of a body cavity.

### RELATED APPLICATIONS

This application claims priority to U.S. Provisional Application No. 63/335,790, filed Apr. 28, 2022, entitled “Flexible Surgical Halo”, the disclosure of which is incorporated by reference herein in its entirety.

### BACKGROUND

Many surgeries today are performed using minimally invasive surgery techniques. Minimally invasive surgery techniques may eliminate any need for open surgery or even for large incisions in a patient's skin. One such minimally invasive technique includes insertion of a hollow tube called a cannula or surgical port into a patient's body cavity where an operation is being performed. Once inserted, the surgical port offers an opening into the body cavity that may be used and reused without causing further damage to the patient's body, such as to insert and remove tools and devices to be used as part of the surgery, to remove tissue located within the body cavity, and so forth. Minimally invasive techniques allow a surgeon to operate instruments from outside of the body to manipulate tissues or other objects within the body. While the surgical port provides an entrance to the body cavity that is large enough for certain instruments, tools, or devices, the surgical port is generally too small to provide vision within the body cavity. In order to accurately guide and manipulate the instruments within the body cavity, an endoscope is utilized that is small enough to enter the body cavity via the surgical port, and an output of the endoscope allows the surgeon to visualize the area inside of the body cavity along with operative portions of the instruments inside the body cavity.

A camera inserted into the body cavity (e.g., a camera as part of an endoscope), however, is vulnerable to becoming soiled, obstructed, or otherwise impaired, such as if body tissues, blood, or other body fluids attach to a lens of the camera and obscure vision of the camera. When vision of the camera is impaired, surgery may be paused until unobstructed camera visibility is reobtained. A common problem that may occur during surgical procedures is for blood to drip down from a patient's body wall where a surgical port is placed, dripping down along the surgical port until it reaches a shaft of the camera, and then flowing down the shaft of the camera and obscuring a camera lens at the end of the shaft of the camera. Pausing surgery to clean the camera frequently occurs multiple times during a surgical process, extending a duration of the surgical procedure. Longer surgery times increase the anesthesia times for the patient and a likelihood of infection, increasing risk of harm to the patient. This is particularly problematic if the camera becomes obscured during a critical portion of the surgical procedure, for example, while a surgeon is manipulating tissue involving major arteries. In this scenario, even a brief pause or disruption to the surgery can cause serious harm to the patient.

Conventional techniques used to maintain camera visibility in a body cavity, however, are faced with numerous challenges that fail to remedy the source of visual impairments. In one example, conventional techniques to maintain camera visibility include removing the camera from the body cavity, cleaning the camera outside of the body cavity, and reintroducing the cleaned camera back into the body cavity. In another example, conventional techniques to maintain camera visibility include a ‘wiper’ mechanism for use within the body cavity, such that the wiper mechanism physically displaces and removes material from the surface of the camera lens. Accordingly, conventional techniques to maintain camera visibility pertain to cleaning and removing material from a camera after it has already become soiled. These conventional techniques are reactive to the camera becoming soiled and are at best a temporary solution, as they do not address the source or cause of the material soiling the camera, and thus the lens continually becomes soiled again and requires repeated interruptions and pauses while the conventional techniques are repeatedly performed.

### SUMMARY

Tools and techniques for a flexible surgical halo are described. These techniques may be utilized, for example, to reduce flow or dripping of blood within a body cavity such as to eliminate or reduce movement of blood along a cannula inserted into a body cavity or along tools or instruments located within a body cavity.

A flexible surgical halo, for instance, may include an elastically deformable body including a tube portion and a canopy portion. The tube portion is configured for attachment to an outer surface of a cannula, and the canopy portion is configured to route liquids along an upper surface or an outer edge of the canopy. The canopy portion has a lower surface configured to ensure that liquid remains on the upper surface or the outer edge (or drips off of the outer edge), without reaching the tube portion. The surgical halo is elastically deformable, which allows it to be deformed during entry into a body cavity (e.g., to reduce its cross-sectional area, allowing for entry via an incision or cannula that has a smaller cross-sectional area than the surgical halo in a dormant state) and return to its original configuration once inside the body cavity. The surgical halo may include other components, such as a marker component (e.g., a radiopaque marker) to enable visibility of the surgical halo when imaging techniques are used on the body, or a retrieval component to facilitate removal of the surgical halo from the body cavity.

This summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures may be indicative of one or more entities and thus reference may be made interchangeably to single or plural forms of the entities in the discussion.

FIG. 1 depicts an illustration of an environment in an example implementation that is operable to employ flexible surgical halo techniques described herein in accordance with one or more embodiments.

FIG. 2 is an illustration of an example cannula that is operable to employ flexible surgical halo techniques described herein in accordance with one or more embodiments.

FIG. 3 is an illustration of an example cannula and an example trocar that are operable to employ flexible surgical halo techniques described herein in accordance with one or more embodiments.

FIG. 4 depicts an illustration of an environment in an example implementation in which cannulas are inserted through a body wall in order to employ flexible surgical halo techniques described herein in accordance with one or more embodiments.

FIG. 5 is an illustration of an example flexible surgical halo that is operable to employ techniques described herein in accordance with one or more embodiments.

FIG. 6 is an illustration of an example flexible surgical halo that is operable to employ techniques described herein in accordance with one or more embodiments.

FIG. 7 is an illustration of an example flexible surgical halo that is operable to employ techniques described herein in accordance with one or more embodiments.

FIG. 8 is an illustration of an example flexible surgical halo attached to a cannula that is operable to employ techniques described herein in accordance with one or more embodiments.

FIG. 9 is an illustration of an example flexible surgical halo attached to a cannula that is operable to employ techniques described herein in accordance with one or more embodiments.

FIG. 10 is an illustration of an example scenario in which a flexible surgical halo is inserted into a body cavity and attached to a cannula in accordance with one or more embodiments.

FIG. 11 is an illustration of an example scenario in which a flexible surgical halo attached to a cannula and inserted into a body cavity in accordance with one or more embodiments.

FIG. 12 is an illustration of an example scenario in which a flexible surgical halo including a retrieval component is in a body cavity in accordance with one or more embodiments.

### DETAILED DESCRIPTION

### Overview

In conventional techniques for maintaining surgical tool cleanliness inside of a body cavity, surgical tools become soiled and the conventional techniques reactively clean the tools. For instance, blood flows or drips from a wall of a body cavity onto surgical implements (e.g., a camera lens of an endoscope) located below the wall. Conventional techniques allow the blood to flow onto the surgical implements, at which point conventional techniques are applied to remove the blood from the surgical implements. Thus, many conventional techniques for maintaining surgical tool cleanliness inside of a body cavity have been found to be unable to counteract a source of contamination, as and operate reactively subsequent to a tool becoming soiled.

Accordingly, flexible surgical halo tools and techniques are described. In one example, a flexible surgical halo includes an elastically deformable body that is attached to a cannula. The flexible surgical halo shields the cannula from becoming soiled, acting similar to an umbrella in routing liquids to an outer edge and protecting components underneath the flexible surgical halo.

The flexible surgical halo, for instance, includes an elastically deformable body including a tube portion and a canopy portion. The tube portion is configured for attachment to an outer surface of a cannula, and the canopy portion is configured to route liquids along an upper surface or an outer edge of the canopy. The canopy portion has a lower surface configured to ensure that liquid remains on the upper surface or the outer edge (or drips off of the outer edge), without reaching the tube portion. The surgical halo is elastically deformable, which allows it to be deformed during entry into a body cavity (e.g., to reduce its cross-sectional area, allowing for entry via an incision or cannula that has a smaller cross-sectional area than the surgical halo in a dormant state) and return to its original configuration once inside the body cavity. The surgical halo may include other components, such as a marker component (e.g., a radiopaque marker) to enable visibility of the surgical halo when imaging techniques are used on the body, or a retrieval component to facilitate removal of the surgical halo from the body cavity. In another example, a cannula includes a flexible surgical halo. When the cannula is inserted through a wall of a body cavity, the attached surgical halo may deform to reduce its cross-sectional area. For instance, the surgical halo in a dormant configuration has a canopy with a curvature that slopes downward (e.g., a top surface forms a convex curve and a bottom surface forms a concave curve). During insertion through a wall, the canopy may deform and invert to instead be directed upward. After insertion, the canopy returns to the dormant configuration inside of the body cavity. The cannula may then be removed from the wall, and the canopy may deform to increase the downward curve (e.g., to be directed downward with a curve approaching parallel with a longitudinal axis). In this way, flexible surgical halo tools and techniques provide the ability to shield a surgical instrument from becoming soiled inside of a body cavity.

In the following discussion, example flexible surgical halo tools are described that may employ the techniques described herein. Example scenarios are described in which the example flexible surgical halo is utilized to shield portions of a cannula (and instruments protruding from the cannula) from liquids or other sources of becoming soiled. Performance of the example scenarios is not limited to the example flexible surgical halo tools, and the example flexible surgical halo tools are not limited to performance of the example scenarios.

### Flexible Surgical Halo

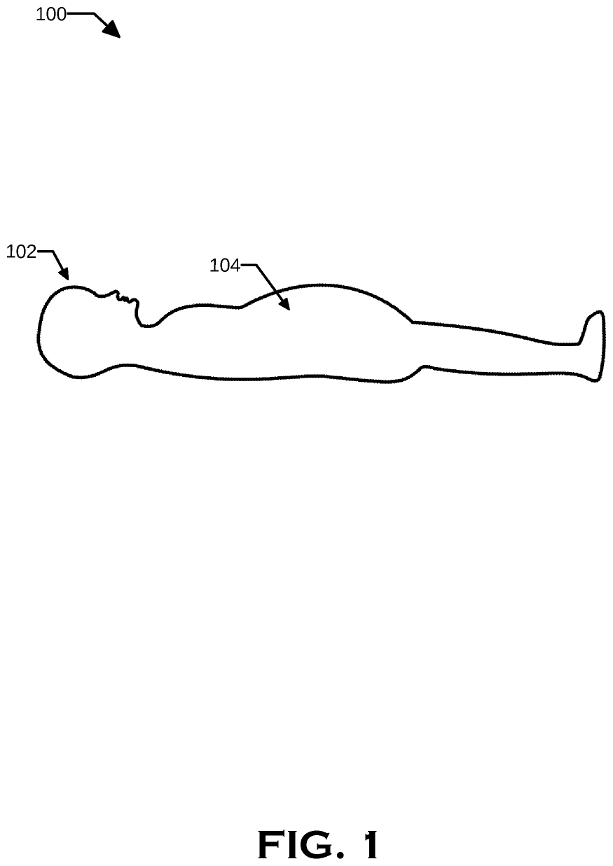

FIG. 1 is an illustration of an environment 100 in an example implementation that is operable to employ the flexible surgical halo device and techniques described herein. The environment 100 includes an example body 102. The body 102 includes a body cavity 104 in which surgery is to be performed. The body 102 is generally illustrated and described with respect to a human body, however it is to be appreciated that the body 102 is representative of any body, such as the body of an animal receiving surgery performed by a veterinarian. The body cavity 104 is a space within the body 102, such as a fluid-filled space inside the body that holds and protects internal organs. The body 102 may include multiple body cavities 104, such as different spaces that are separated by membranes or other structures. For example, the body cavity 104 may be an abdominal cavity, an abdomino-pelvic cavity, a pelvic cavity, a ventral cavity, a thoracic cavity, a dorsal cavity, a spinal cavity, a cranial cavity, and so forth. The body cavity 104 may be any space within the body 104, including spaces that are artificially created or enlarged such as through the infusion of a gas (e.g., carbon dioxide) into the body 104. In an example minimally invasive surgery technique, a small incision is created through a body wall (e.g., skin, tissues, and so forth) of the body 102, and a surgical port is established (e.g., via insertion of a trocar and a cannula into the incision) to provide a physical opening between outside the body 102 and inside the body cavity 104.

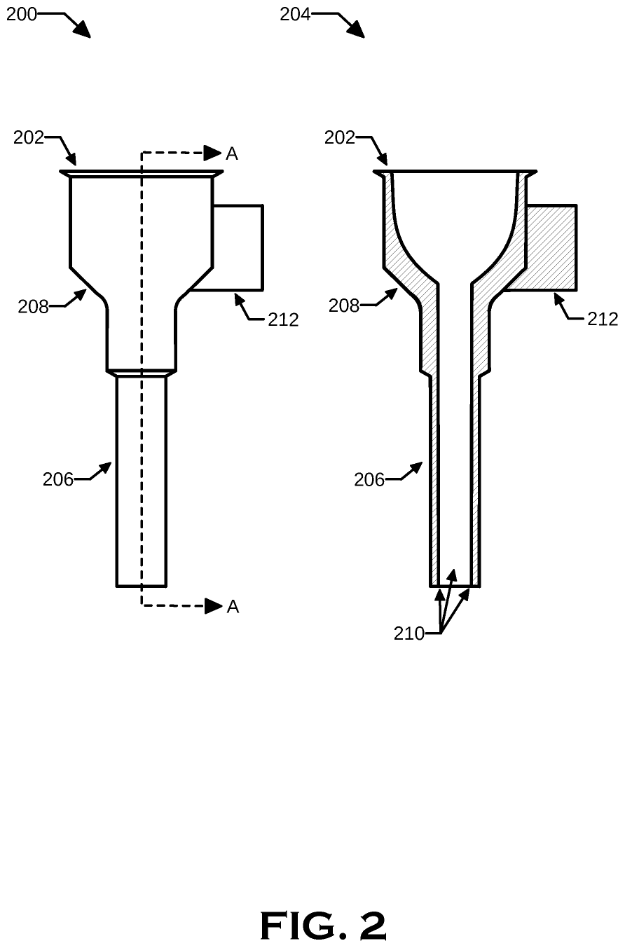

FIG. 2 depicts an example scenario 200 including an example cannula 202 and an example scenario 204 including the cannula 202 from a side view with a cross section taken along line A-A of the environment in the scenario 200. The cannula 202 includes a cannula rod 206 and a cannula head 208. The cannula rod 206 has an inner surface 210 with a radius forming a hollow tube longitudinally through the cannula rod 206. This hollow tube defined by the inner surface 210 functions as an opening for use by other instruments such as graspers, cameras, scissors, staplers, and so forth. In an example, the cannula rod 206 may be placed in an incision in a body, and the hollow tube provides an opening between a body cavity in the body and space external to the body.

In implementations, the cannula 202 includes a cannula mount 212. The cannula mount 212 includes functionality to attach the cannula 202 to another device, such as a manipulator arm of a patient side cart, a mechanical arm of a robotic surgical device, and so forth. The cannula mount 212 may be, for instance, a projection configured to be inserted into a corresponding recess on another device, may include holes, tabs, or rods designed to interface with functionalities on another device, and so forth.

Although the cannula rod 206 is depicted as being straight, it is to be appreciated that the cannula rod 206 may take other forms, such as a curved rod, a flexible rod, a straight rod with tapered sides, and so forth. In example implementations, the inner surface 210 corresponds to a standard diameter of medical cannulas, such as approximately 5 mm, 6 mm, 8 mm, 10 mm, 12 mm, and so forth.

In implementations, the cannula 202 is made as a reusable cannula (such as a cannula configured for use with a robotic surgical device) from a durable and washable material such as stainless steel. In other implementations, the cannula 202 is made as a single-use or disposable cannula, and may be formed from various types of plastics or polymers, and so forth.

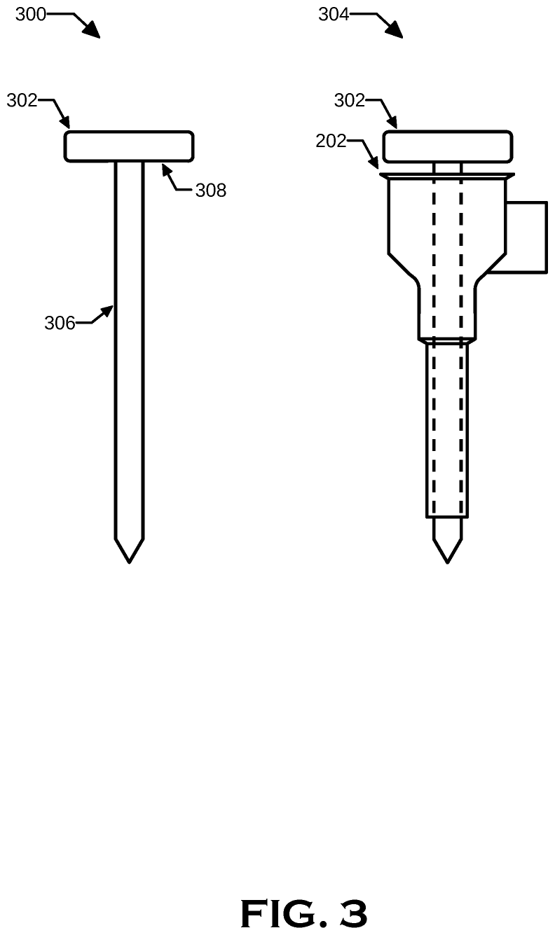

FIG. 3 depicts an example scenario 300 including an example trocar 302, and an example scenario 304 in which the example trocar 302 is placed within the cannula 202 of FIG. 2. The trocar 302 includes a trocar rod 306 and a trocar head 308. In implementations, the trocar rod 306 has a pointed end that is tapered to be narrowest at a tip opposite the trocar head 308. The trocar rod 306 is generally configured to have an outer surface with a radius from a longitudinal axis. In implementations, the radius of the trocar rod 306 is similar in dimension to the inner surface 210 of the cannula 202 of FIG. 2. For instance, the radius of the trocar rod 306 is dimensioned to be as close to the radius of the inner surface 210 as possible while remaining smaller than the inner surface 210 and accounting for tolerance errors.

The example scenario 304 depicts the trocar 302 within the cannula 202. The trocar rod 306 is inserted into, and extends through, the hole formed by the inner surface 210. For instance, the tip of the trocar rod 306 extends beyond a bottom of the cannula 202, while the trocar head 308 remains above the top of the cannula 202. In this way, the trocar 302 and the cannula 202 collectively create a near-seamless form, such that little or no extraneous material is able to be located or inserted between the trocar rod 306 and the inner surface 210. This allows the trocar 302 and the cannula 202, when configured together, to puncture material, be pushed through an incision in material, and so forth without allowing the material or portions of the material entry into the cannula 202.

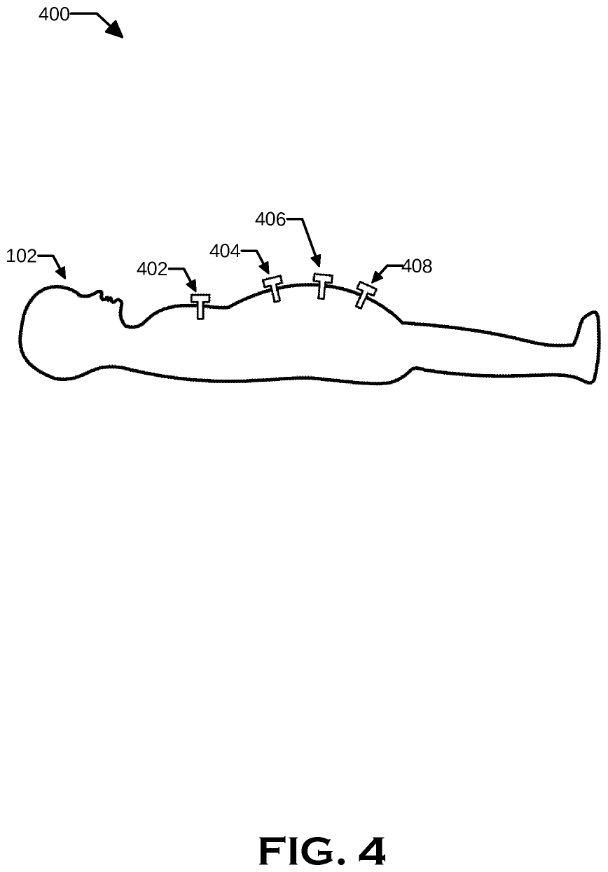

FIG. 4 depicts an example environment 400 that includes the body 102 of FIG. 1 and cannulas 402, 404, 406, and 408. Each of the cannulas 402, 404, 406, and 408, for instance, correspond to a respective cannula 202 of FIG. 2. In this example, respective trocars were inserted into the cannulas 402, 404, 406, and 408, and the respective cannula have been inserted into the body 102 (e.g., by inserting the cannula and trocar combinations into respective incisions in the body 102). The cannulas may be inserted into different respective body cavities of the body 102 or may each be inserted into a same body cavity of the body 102. Once a cannula is inserted into the body 102 (e.g., such that a portion of the cannula rod is inside a wall of the body 102, the tip of the cannula rod is inside the body cavity, and the cannula head is outside the body), the trocar is removed from the cannula. In this way, the cannulas 402, 404, 406, and 408 remain embedded in the body 102 without their respective trocars, allowing the respective hollow tubes defined by the inner surfaces of the cannulas 402, 404, 406, and 408 to function as an opening into the body cavity.

Accordingly, the cannulas 402, 404, 406, and 408 each individually provide a respective opening which may be utilized by a surgeon to insert instruments used in a surgical procedure, such as a camera (e.g., an endoscopic camera with a long, flexible shaft with a camera lens at the tip of the shaft), graspers, scissors, suction tips, needle holders, sutures, and so forth.

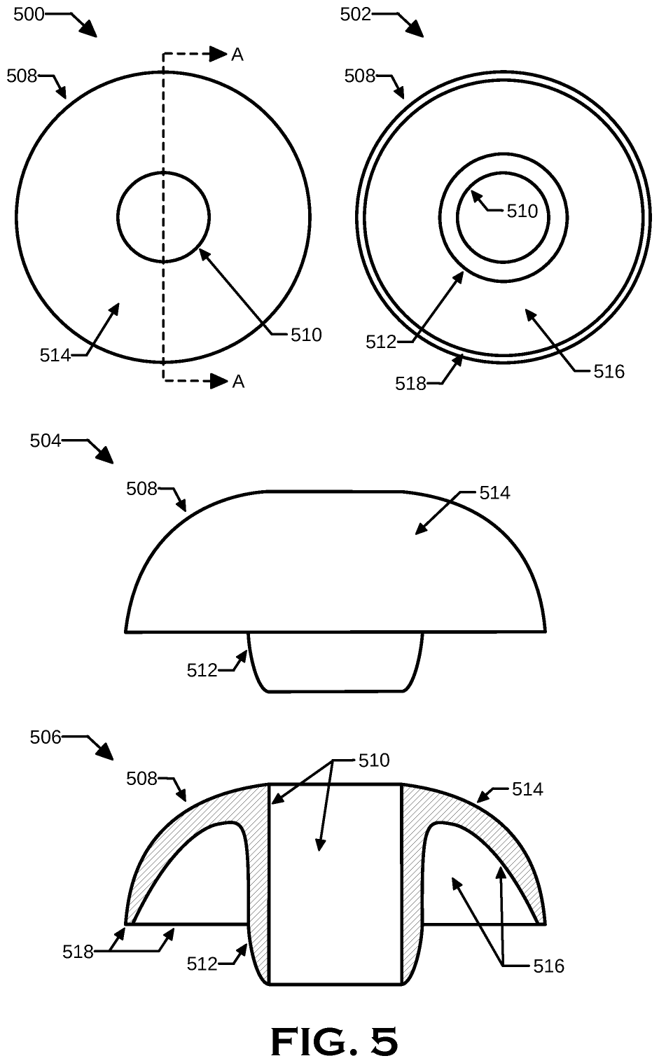

FIG. 5 depicts example environments 500, 502, 504, and 506 that each include a surgical halo 508. Example environment 500 depicts the surgical halo 508 from a top view. Example environment 502 depicts the surgical halo 508 from a bottom view. Example environment 504 depicts the surgical halo 508 from a side view. Example environment 506 depicts the surgical halo 508 from a side view with a cross section taken along line A-A of environment 500.

The surgical halo 508 is made of a deformable material configured to retain a particular shape or configuration when no external forces are applied (e.g., while the surgical halo is in a dormant state), deform from the particular shape or configuration into a new shape or configuration when external forces are applied (e.g., while the surgical halo is in a deformed state), and return to the particular shape or configuration when the external forces are no longer applied. For instance, the surgical halo 508 is elastically deformable. Elastic deformation refers to a temporary deformation of a material's shape that is caused by a force or load, and is self-reversing after removing the force or load to regain an original shape and dimensions, such that the deformation is reversible and non-permanent (e.g., while the force or load is within an elastic limit of the material). In implementations, the surgical halo 508 is a single discrete object molded from a silicone rubber or another elastomer such as thermoplastic elastomers, polyolefin elastomers, polyvinyl chloride, rubber, hydrogels, and so forth that is elastically deformable.

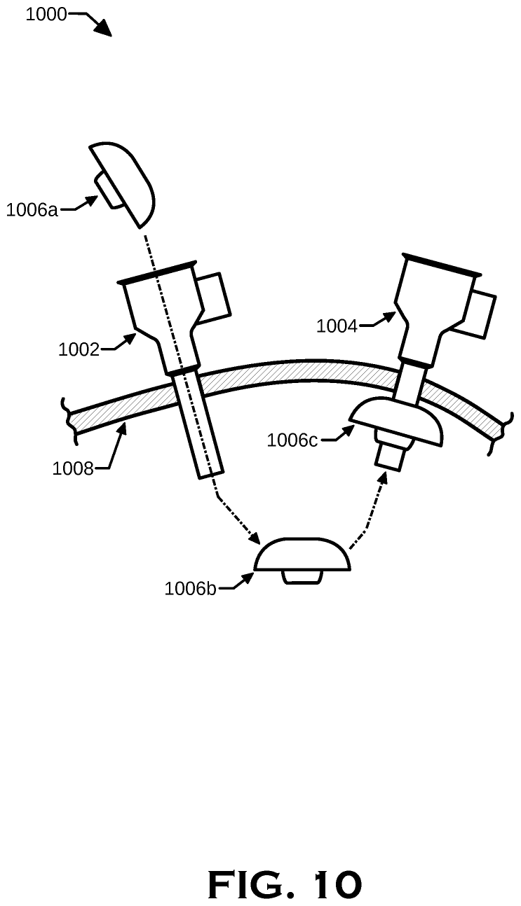

The surgical halo 508 has an interior surface 510 with a radius that defines an opening passing through the surgical halo 508 from top to bottom (e.g., a hole along a longitudinal axis). The surgical halo 508 is capable of deformation in both size and shape, including deformation of the size or shape of the interior surface 510. In implementations, the surgical halo 508 is configured to both be deformed to fit through an inner radius of a cannula, (e.g., corresponding to the inner surface 210 of the cannula 202 of FIG. 2), and to be deformed to fit around an outer surface of the same cannula. For example, although the surgical halo 508 in a dormant state has dimensions too large to fit within the inner radius of the cannula, the surgical halo 508 may be deformed (such as when subjected to an external force) to fit within the inner radius of the cannula. This allows, for instance, the surgical halo 508 to be inserted into a body cavity through the cannula while in a deformed state, and return to the dormant state once within the body cavity (e.g., when the external force is removed from the surgical halo 508), as described below with respect to FIG. 10.

The surgical halo 508 has an exterior surface 512, an upper surface 514, and a lower surface 516. A tube along a longitudinal axis is defined by a region between the interior surface 510 and the exterior surface 512. In implementations, the tube has a generally uniform thickness (e.g., the interior surface 510 and the exterior surface 512 are parallel). In other implementations, the tube has a tapered shape, such that a thickness at an end of the tube opposite the upper surface 514 and the lower surface 516 is less than a thickness of another portion of the tube. As illustrated in the environments 504 and 506, an end of the tube has a curved taper while a middle of the tube has a uniform thickness, and a top of the tube has an increased thickness proximate its connection with the lower surface 516.

A canopy is defined by a region between the upper surface 514 and the lower surface 516. The upper surface 514 has a convex curve from a top point (e.g., where the upper surface 514 intersects with the exterior surface 512, or where the exterior surface 512 would be if not for the presence of the canopy), to an outer edge 518 (e.g., where the upper surface 514 connects to the lower surface 516), and extends radially from the longitudinal axis such that the canopy partially encircles the interior surface 510. The lower surface 516 has a concave curve from the outer edge 518 to an upper point (e.g., where the lower surface 516 connects to the exterior surface 512). In this way, the lower surface 516 extends radially from the longitudinal axis such that a portion of the canopy encircles a portion of the exterior surface 512 without contacting between the respective portions, e.g., to create a void between the portion of the canopy and the portion of the exterior surface 512. The canopy may be, for example, a shell of a shape generally corresponding to a spherical cap or spherical dome (e.g., a portion of sphere cut off by a plane).

The configuration with a void between portions of the lower surface 516 and portions of the exterior surface 512 provides several benefits. For instance, the surgical halo 508 may be deformed to remove the void (e.g., such that the portions of the lower surface 516 contact the exterior surface 512), thus decreasing an overall radius of the surgical halo 508 with respect to the longitudinal axis. The surgical halo 508 may also be deformed to fold the canopy ‘upward’, generally inverting a shape of the canopy. For example, the canopy may be deformed into a deformed state where the upper surface 514 has a concave curve (as opposed to the convex curve in the dormant state) from the top point to the outer edge and the lower surface 516 has a convex curve (as opposed to the concave curve in the dormant state) from the outer edge 518 to an upper point (e.g., where the lower surface 516 connects to the exterior surface 512). In this example deformed state, for instance, the lower surface 516 is radially further from the longitudinal axis than the upper surface 514.

The configuration with a void between portions of the lower surface 516 and portions of the exterior surface 512 further serves to provide geometries with which to control and route a liquid that comes into contact with the surgical halo 508. In a vertical or upright position (e.g., as illustrated in the environments 504 and 506 with the longitudinal axis aligned with a direction of gravity and with the upper surface 514 located higher than the lower surface 516 in the dormant state), every portion of the upper surface 514 and the lower surface 516 is higher than the outer edge 518. Accordingly, any liquid on the upper surface 514 or the lower surface 516 will be routed, by gravity, to the outer edge 518. For every orientation of the surgical halo 508 within 90 degrees from upright with respect to the longitudinal axis, at least a portion of the outer edge 518 is located lower than all portions of the upper surface 514 and the lower surface 516. Accordingly, for any orientation with up to a 90 degree tilt, liquid on the canopy of the surgical halo 508 is routed with gravity toward a lowest point of the outer edge 518.

In scenarios where the surgical halo 508 is rotated from an upright position, a portion of the outer edge 518 may be located higher than a portion of the lower surface 516. However, the surgical halo 508 is shaped such that the combination the curvature of the lower surface 516 combined with the radial configured of the outer edge 518 ensures that for any point on the outer edge 518 (aside from a bottommost point), an adjacent point on the outer edge 518 is lower than any adjacent points on the lower surface 516. In this way, when access to the interior surface is blocked (e.g., the surgical halo 508 is placed on cannula such that the cannula fills the radius defined by the inner surface 510), any liquid on the upper surface 514 or the outer edge 518 is routed via gravity to continue along either the upper surface 514 or the outer edge 518 until it reaches a bottommost point of the outer edge 518, and gravity will not route liquid onto the lower surface 516. Accordingly, the canopy serves to shield the exterior surface 512 from liquid moving due to gravity, and by extension, shields an object protruding from a bottom end of the surgical halo 508 (e.g., a cannula and any cameras or other surgical implements placed through the cannula).

In implementations, the outer edge 518 defines points of intersection between the upper surface 514 and the lower surface 516. However, in other implementations the outer edge 518 represents a surface between the upper surface 514 and the lower surface 516. In this way, the canopy may have a minimum thickness, e.g., a minimum thickness determined by a width of the outer edge 518. By incorporating a minimum thickness, durability of the surgical halo 508 is increased and a likelihood of damage to the surgical halo 508 is decreased. The outer edge 518 may be a flat surface, but may also be a curved or rounded portion of the canopy that connects the upper surface 514 and the lower surface 516 (e.g., a bullnose edge formed in-between the upper surface 514 and the lower surface 516).

In some implementations, a height of the tube of the surgical halo 508 (e.g., measured along the longitudinal axis) exceeds a height of the canopy, such as in the examples illustrated in environments 504 and 506. However, other implementations of the surgical halo 508 include a height of the tube that equals a height of the canopy, or a height of the tube that is less than a height of the canopy. The surgical halo 508 may be configured such that a portion of the tube may be removed (e.g., by cutting the tube with a scalpel, scissors, and so forth) to alter a height of the tube without compromising a structure of the remaining portions of the surgical halo 508.

The surgical halo 508, in implementations, may further include a marker component. The marker component is configured to provide visibility of the surgical halo 508 under imaging techniques outside of the visible spectrum of light. For example, the marker component is a radiopaque marker that is opaque to X-rays or other radiation, thereby blocking x-rays during x-ray imaging techniques and forming a distinct and visible indicator of the marker component in resultant x-ray image. The marker component may include, for example, radiopaque contrasting agents such as barium sulfate, diatrizoate, iohexol, iothalamate, and so forth.

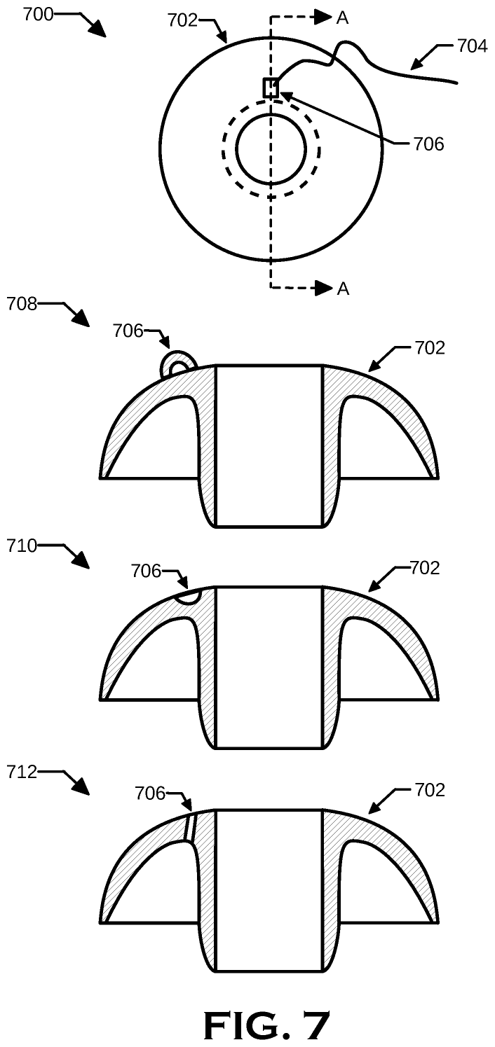

In implementations, the marker component is included within a material used to form the surgical halo 508 itself, such as through the inclusion of barium sulfate into a silicone rubber that forms the surgical halo 508. In other implementations, the marker component is a discrete object embedded within the surgical halo 508 or otherwise attached to the surgical halo 508, such as a radiopaque string attached to the surgical halo 508 or embedded within the surgical halo 508. A radiopaque string, for example, may include nylon filaments with a radiopaque coating, polyester filaments containing platinum wires, silk fibers cross linked with DMDF (2,5-dimethoxy-2,5-dihydrofuran) and iodine, and so forth. The marker component may be included, for instance, as part of a retrieval component attached to the surgical halo 508 as described in greater detail with respect to FIG. 7. Through inclusion of the marker component, the surgical halo 508 may be easily located within a body via imaging techniques in the event that the surgical halo 508 becomes misplaced within the body or otherwise needs locating within the body.

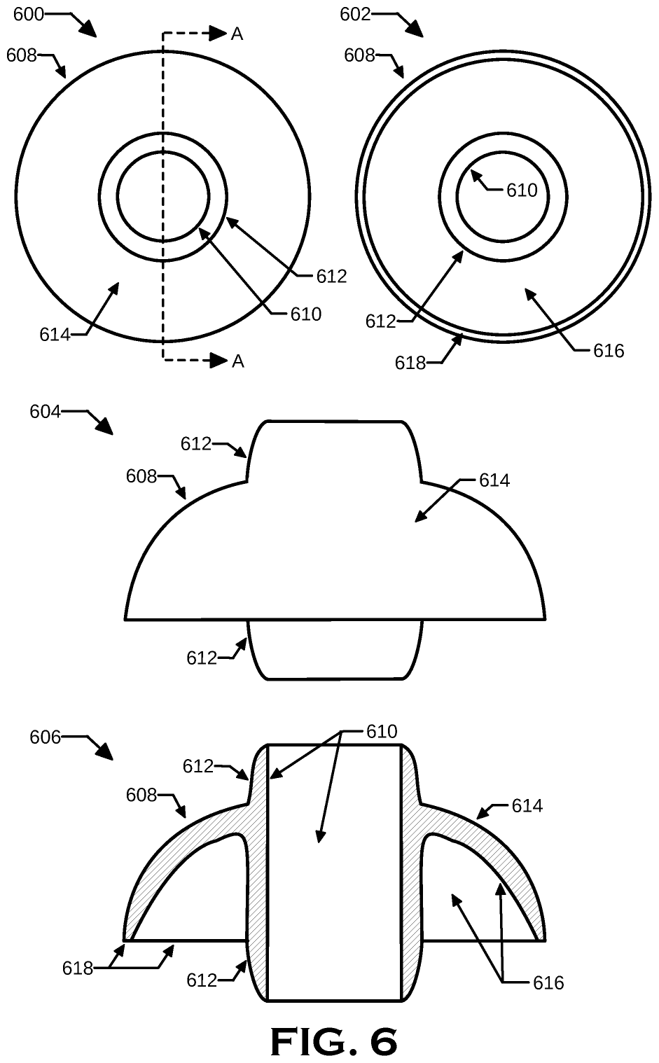

FIG. 6 depicts example environments 600, 602, 604, and 606 that each include a surgical halo 608. Example environment 600 depicts the surgical halo 608 from a top view. Example environment 602 depicts the surgical halo 608 from a bottom view. Example environment 604 depicts the surgical halo 608 from a side view. Example environment 606 depicts the surgical halo 608 from a side view with a cross section taken along line A-A of environment 600.

The surgical halo 608 is similar to the surgical halo 508 of FIG. 5. While the surgical halo 508 includes a canopy that is generally aligned with a top of the tube, the surgical halo 608 includes a canopy that is aligned with a middle portion of the tube. Accordingly, the surgical halo 608 includes an interior surface 610, an exterior surface 612, an upper surface 614, a lower surface 616, and an outer edge 618, that each correspond, respectively, to the interior surface 510, the exterior surface 512, the upper surface 514, the lower surface 516, and the outer edge 518 of FIG. 5. However, the interior surface 610 and the exterior surface 612 each extend beyond a top of the canopy. For example, the ‘tube’ portion of the surgical halo 806 is clearly visible (e.g., both the interior surface 610 and the exterior surface 612 are distinct from the canopy) in both top view 600. The surgical halo 508 of FIG. 5, in contrast, has a smooth transition between the canopy and a top of the tube, such that the exterior surface 512 is not distinctly visible from the top view 500. In implementations, the tube portion is tapered in two directions respective to where the canopy is attached to the tube. For instance, the tube portion is thickest where the canopy is attached to the tube and thinnest at the ends of the tube.

FIG. 7 depicts an example scenario 700 which illustrates an example surgical halo 702 (e.g., the surgical halo 508 of FIG. 5) that includes a retrieval component 704. The retrieval component 704 is configured to provide a structure with which the surgical halo 702 may be grasped or manipulated. For example, the retrieval component 704 may be a length of suture such as silk or polyester suture, for example a 24 inch tail of #2 silk suture. In implementations, the retrieval component 704 includes a marker component (e.g., the marker component as described with respect to FIG. 5). For example, the retrieval component 704 is a radiopaque string or suture.

In implementations, the surgical halo 702 includes a retrieval anchor 706. The retrieval anchor 706 provides a structure for use in affixing the retrieval component 704 to the surgical halo 702. The retrieval anchor 706 may take various forms, examples of which are included in example scenarios 708, 710, and 712.

Example scenario 708 depicts an example surgical halo 702 from a side view with a cross section taken along line A-A of environment 700. In this example, the retrieval anchor 706 is configured as a “loop” that protrudes from the upper surface of the canopy of the surgical halo 614. The retrieval component 704 may be inserted through the hole formed by the loop of the retrieval anchor 706 and tied to the retrieval anchor 706 or otherwise fastened back upon itself. Another example of a retrieval anchor 706 protruding from the upper surface of the canopy of the surgical halo 702 includes a protruding form (e.g., of a generally cylindrical shape) with concave sides, such that the retrieval component 704 may be tied around a narrowest portion of the concave sides of the protruding form.

Example scenario 710 depicts an example surgical halo 702 from a side view with a cross section taken along line A-A of environment 700. In this example, the retrieval anchor 706 is configured as an excavated torus segment that forms a cavity within the canopy of the surgical halo 702. In this example, the retrieval anchor 706 is a rod corresponding to the center of the torus (e.g., a rod that extends above and across the cavity). The retrieval component 704 may be inserted into the cavity and looped around the rod.

Example scenario 712 depicts an example surgical halo 702 from a side view with a cross section taken along line A-A of environment 700. In this example, the retrieval anchor 706 is configured as an excavated channel within the canopy of the surgical halo 702 that provides an opening between the upper surface and the lower surface of the canopy. The retrieval component 704 may be inserted through the channel, looped around the tube of the surgical halo, and inserted back through the channel. In doing so, any force exerted on the retrieval component 704 is exerted against the tube of the surgical halo as opposed to the retrieval anchor 706, which provides increased strength and durability and reduces a likelihood of damage to the surgical halo 702 compared to the retrieval anchors 706 described with respect to example scenarios 708 and 710. In implementations, multiple channels are provided in the structure of the surgical halo 702, such that different portions of the retrieval component 704 pass through different respective channels. For example, the surgical halo 702 may have an ‘entry’ channel and an ‘exit’ channel, such that different channel may be utilized each time the retrieval component 704 passes through the surgical halo 702. In implementations, the channels may be created during a process of attaching the retrieval anchor 706, such as by passing the retrieval component 704 through the surgical halo 702 with use of a needle.

The surgical halo 702, in implementations, includes the retrieval component 704 but not the retrieval anchor 706. In one example, the retrieval component 704 may be tied around a portion of the tube above the canopy (e.g., around a portion of the exterior surface 612 of FIG. 6 that is above the upper surface 614. In another example, the retrieval component 704 is partially embedded within the body of the surgical halo 702. For instance, in an example where the surgical halo 702 is formed via a molding process, an end portion of the retrieval component 704 is placed within the mold such that it becomes embedded within the surgical halo 702 during the molding process itself or before a material forming the surgical halo 702 solidifies. In this way, the retrieval component 704 may be permanently attached to the surgical halo 702 without altering the overall dimensions of the surgical halo 702 or including a retrieval anchor 706.

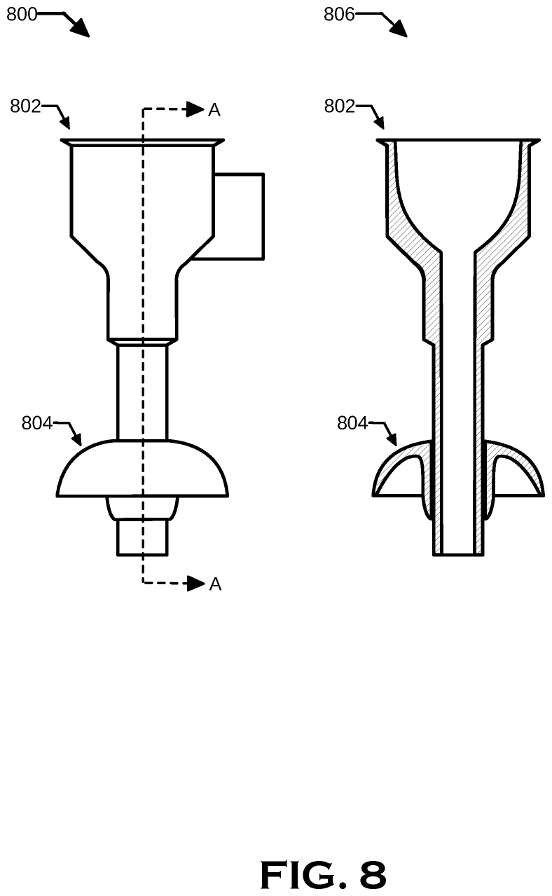

FIG. 8 depicts an example scenario 800 including an example cannula 802 that includes a surgical halo 804, and an example scenario 806 including the cannula 802 from a side view with a cross section taken along line A-A of the environment in the scenario 800. In implementations, the surgical halo 804 is a discrete object removably coupled with the cannula 802 (e.g., as generally described with respect to FIGS. 5-7). In other implementations, the cannula 802 and the surgical halo 804 are both parts of a same device. For instance, the surgical halo 804 is affixed to the cannula 802 such that they are manipulable as a single object and do not move with respect to one another. In an example implementation as a single object, the surgical halo 804 affixed to the cannula 802 does not include a retrieval component or a marker component, as the cannula 802 itself serves as a retrieval component (e.g., removing the cannula 802 from a body cavity also removes the attached surgical halo 804) and the cannula 802 does not pose a risk of being misplaced in a body cavity as it does not fully enter the body cavity (e.g., a port head of the cannula 802 has dimensions greater than those of an incision the cannula 802 is placed through.

In implementations, an inner surface of the surgical halo 804 has a radius lesser than a radius of an outer surface of the cannula rod of the cannula 802 while the surgical halo 804 is in a dormant state (e.g., prior to being placed on the cannula rod). As the presence of the cannula rod prevents the inner surface of the surgical halo 804 from returning to the dormant state, the surgical halo 804 continually exerts a compressive force inward against the cannula rod. This compressive force provides a normal force (perpendicular to the longitudinal axis) between the surgical halo 804 and the cannula rod, thus providing a friction force that acts against movement of the surgical halo 804 with respect to the cannula rod.

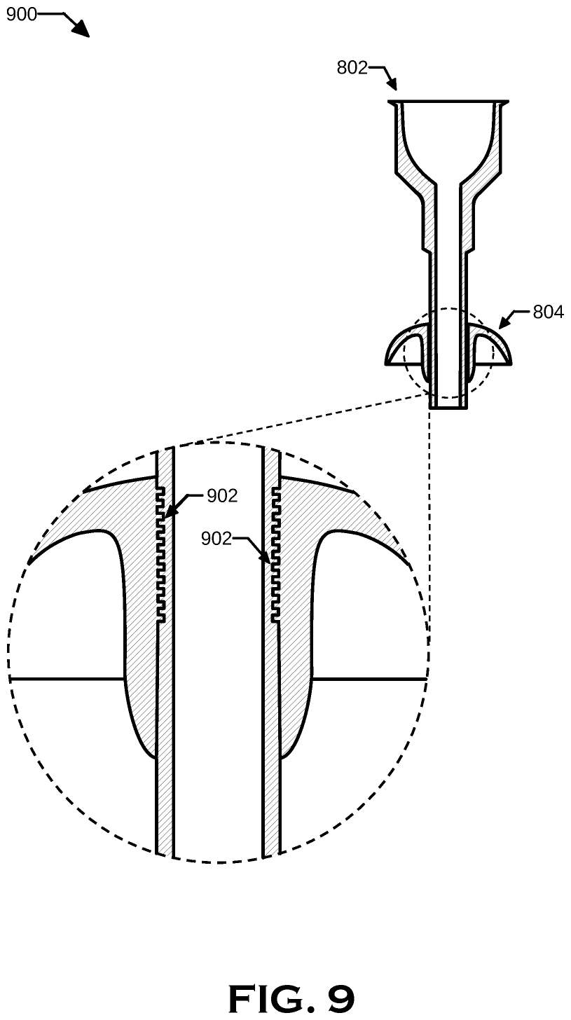

In implementations, additional functionalities are utilized to further affix the surgical halo 804 to the cannula 802. For instance, the cannula 802 and the surgical halo 804 may be molded as a single unit, adhesive may be used between the cannula 804 and the surgical halo 804, and so forth. In an example, the cannula 804 includes grooves or protrusions in the cannula rod, such that the inner surface of the surgical halo 804 forms a non-linear shape (from the perspective of a side view) while on the cannula 804, as described in greater detail with respect to FIG. 9.

FIG. 9 depicts an example scenario 900 including the example cannula 802 of FIG. 8 and surgical halo 804 from a side view with a cross section taken along line A-A of the environment in the scenario 800, and includes an enlarged illustration of a portion of a cannula shaft of the cannula 802 and the surgical halo 804. In this example, the cannula 802 includes grooves 902. The grooves can take a variety of shapes, such as rectangular notches, “T” shaped notches, “L” shaped notches, circular notches, and so forth. In implementations, the grooves may be very small, e.g., less than 1 mm in depth or width. The compressive force exerted by the surgical halo 804 causes portions of the surgical halo 804 to enter the grooves 902, such that a shape of the interior surface of the surgical halo 804 at least partially follows a shape of the grooves 902. This increases adhesion between the cannula 802 and the surgical halo 804. For instance, a force applied parallel to a longitudinal axis of the cannula 802 and the surgical halo 804 is resisted by friction between the cannula 802 and the surgical halo 804, but is also resisted by a normal force parallel to the longitudinal axis by portions of the surgical halo 804 pressing against walls of the grooves 902 that are perpendicular to the longitudinal axis.

FIG. 10 depicts an example scenario 1000 including an example cannula 1002, an example cannula 1004, and an example surgical halo 1006. The cannulas 1002 and 1004 may each, for instance, be a respective cannula 202 of FIG. 2. The cannulas 1002 and 1004 are located partially in a body, such that portions of the cannulas are respectively embedded in portions of a wall 1008 (e.g., a layer of skin and tissue separating a body cavity from outside of the body) such that a first end of the cannula is on a first side of the wall 1008 and an opposite end of the cannula is on an opposite side of the wall 1008. The example surgical halo 1006 is depicted in this example in three different states (e.g., at three different points in time as a process progresses) as 1006a, 1006b, and 1006c, respectively.

The surgical halo 1006a in the first state is in a dormant state on a first side of the wall 1008 (e.g., outside of a body). The surgical halo 1006 is deformed to fit through a passage defined by an interior surface of the cannula 1002, passes through the cannula 1002, and exits the cannula 1002 as depicted with a dotted line from state 1006a to state 1006b. The deformation of the surgical halo 1006 to fit through the passage may include, for instance, compressing the surgical halo 1006 as the dimensions of the surgical halo 1006 in a dormant state may exceed a cross section or radius of the passage. After exiting the cannula 1002, the surgical halo 1006b in the second state has been released from external forces causing deformation, and returns to a dormant state on a second side of the wall 1008 (e.g., inside of a body cavity). Additional external forces are then applied to the surgical halo 1006 to expand an interior surface of the surgical halo 1006, and the surgical halo 1006 is placed around the cannula 1004, as depicted with a dotted line from state 1006b to state 1006c.

In implementations, the surgical halo 1006 is configured with an inner surface in the dormant state having dimensions smaller than an outer surface of the cannula 1004 (e.g., an outside surface of a cannula rod of the cannula 1004). The surgical halo 1006 is deformable to increase the size of the inner surface to exceed a size of the outer surface of the cannula, allowing the surgical halo 1068 to be placed around the outer surface of the cannula (e.g., to be placed on the outer surface of the cannula rod). Once placed on the cannula rod, an external force that caused the deformation to increase a radius of the inner surface is removed, causing the surgical halo 1006 to attempt to return to the dormant state, thus reducing the radius of the inner surface until it equals the outer radius of the cannula, and the surgical halo 1006 enters the third state as surgical halo 1006c. As the presence of the cannula rod prevents the surgical halo 1006c from returning to the dormant state, the surgical halo 1006c continually exerts a compressive force inward against the cannula rod. This compressive force provides a normal force perpendicular to the longitudinal axis between the surgical halo 1006c and the cannula 1004, thus providing a friction force that acts against movement of the surgical halo 1006c with respect to the cannula 1004. In implementations, the friction force is sufficient to couple the surgical halo 1006c in a fixed location with respect to the cannula 1004.

In this way, the surgical halo 1006c maintains a position between the wall 1008 and a bottom of the cannula 1004. Blood or other bodily fluids that drip from the wall 1008 will encounter the surgical halo 1006c, and are routed away from the bottom of the cannula 1004. As discussed above with respect to FIG. 5, liquid on the surgical halo 1006c is unable to reach the bottom of the cannula 1004 so long as the surgical halo 1006c remains oriented within 90 degree from vertical.

Manipulation of the surgical halo 1006, such as to achieve movement and positioning within the body cavity as described above, may be achieved through the use of tools inserted through various cannulas. For example, a camera may be inserted through a cannula to achieve visibility within the body cavity, and graspers inserted through a cannula enable physical manipulation of the surgical halo 1006 within the body cavity. In implementations, a trocar is placed inside of the cannula 1004 to provide a tapered tip to facilitate placement of the surgical halo 1006. For instance, the tapered tip may allow the surgical halo 1006 to expand outward perpendicular to the cannula 1004 based on a force applied parallel to a longitudinal axis of the cannula 1004. Once the surgical halo 1006 is placed around the cannula 1004 and in the third state as surgical halo 1006c, the trocar may be removed from the cannula 1004.

In this way, instruments (e.g., a camera) inserted into the body cavity via cannula 1004 are protected from blood dripping from the wall 1008 of the body cavity, and operation of an instrument may proceed unobstructed and without pausing surgery to clean and maintain the instrument. The process described above may be repeated, such as to place a second surgical halo on the cannula 1002.

In implementations, a retrieval component of the surgical halo 1006 (e.g., the retrieval component 704 of FIG. 7) is configured with a length such that a portion of the retrieval component remains outside of the body. In the scenario described above, for instance, the retrieval component remains partially outside of the cannula 1002, continues through the cannula 1002, and continues through the body cavity and remains connected to the surgical halo 1006c. In this way, the surgical halo 1006 may be retrieved and removed from the body cavity by simply pulling on the portion of the retrieval component that is outside of the body until the surgical halo 1006 exits the body through the cannula 1002.

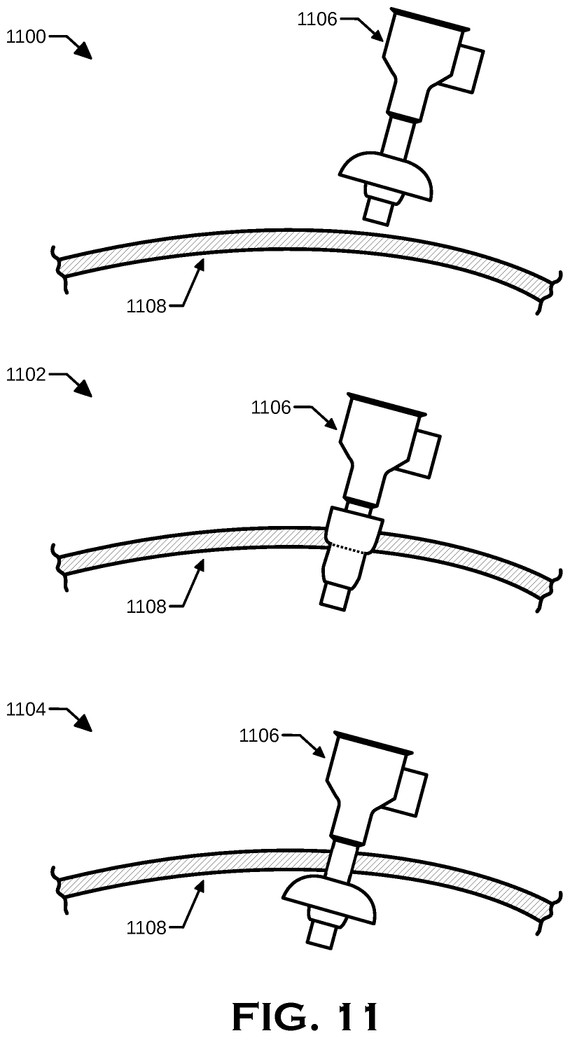

FIG. 11 depicts example scenarios 1100, 1102, and 1104 including an example cannula 1106 that includes a surgical halo. The cannula 1106, for instance, may be the cannula 802 and surgical halo 804 of FIG. 8. The scenarios 1100, 1102, and 1104 depict that cannula 1106 in different states such as corresponding to different points in time as a process progresses.

In the scenario 1100, the cannula 1106 is located outside of a body and on a first side of a wall 1108 (e.g., a layer of skin and tissue separating a body cavity from outside of the body), and a canopy of the surgical halo of the cannula 1106 is in a dormant state.

In the scenario 1102, the cannula 1106 is located partially within the wall 1108 (e.g., within an incision in the wall 1108) such that a first end of the cannula 1106 is on a first side of the wall 1108 and an opposite end of the cannula 1106 is on an opposite side of the wall 1108. In this state, the cannula 1106 is considered ‘in transit’, as it has not fully completed its passage through the wall 1108. The canopy of the surgical halo has been ‘flipped up’ or inverted, such that a lower surface of the canopy faces outward and an upper surface of the canopy faces inward with respect to the longitudinal axis. This deformed state has a reduced cross-sectional area of the surgical halo with respect to the wall 1108. The surgical halo may enter the deformed state based on pressure exerted by the wall 1108 as entry to the wall 1108 commences. For instance, in a dormant state, the canopy of the surgical halo exceeds a cross-sectional area provided by an incision in the wall 1108. As the cannula 1106 is pushed into the incision, the canopy of the surgical halo is pressed downward into the wall 1108. As the cannula 1106 is deformable, the canopy remains against the wall 1108 while the tube of the surgical halo continues progressing through the wall 1108. As a point of intersection between the lower surface of the canopy and the tube reaches the wall 1108, the canopy continues its inversion and is pushed upward until it has a small enough cross-sectional area to enter the wall 1008 (e.g., it may move toward a position of being generally parallel with the tube). In this way, a portion of the canopy that is closest to the tube may enter the wall 1108 before portions of the canopy further from the tube, with the outer edge of the canopy being the last portion of the canopy to enter the wall 1108.

In the scenario 1104, the cannula 1106 has completed its insertion through the wall 1108, and the cannula 1106 is located partially within the wall 1108 such that a first end of the cannula 1106 is on a first side of the wall 1108 and an opposite end of the cannula 1106 is on an opposite side of the wall 1108. The entirety of the surgical halo has passed through the wall and is inside of the body cavity. As the wall 1108 is no longer exerting force upon the surgical halo, the surgical halo returns to its dormant state and ‘flips’ the canopy back to the dormant position through its own internal elastic forces.

In this way, the surgical halo of the cannula 1106 maintains a position between the wall 1108 and a bottom of the cannula 1106. Blood or other bodily fluids that drip from the wall 1108 will encounter the surgical halo, and are routed away from the bottom of the cannula 1106. As discussed above with respect to FIG. 5, liquid on the surgical halo is unable to reach the bottom of the cannula 1106 so long as the cannula 1106 remains oriented within 90 degree from vertical. An instrument (e.g., a camera) inserted into the body cavity via the cannula 1106 is thus protected from blood dripping from the wall 1108 of the body cavity, and operation of the instrument may proceed unobstructed and without pausing surgery to clean and maintain the instrument.

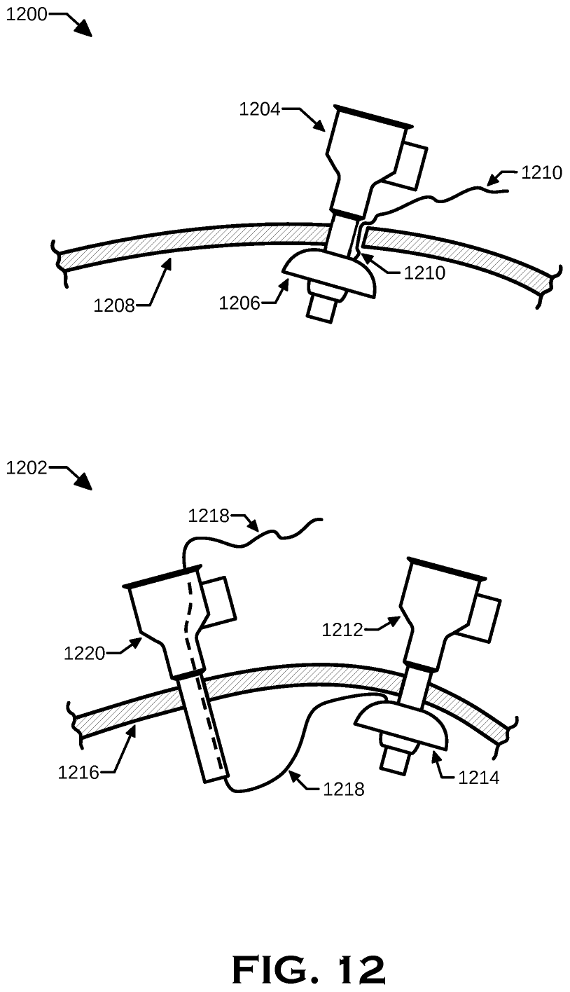

FIG. 12 depicts example scenarios 1200 and 1202 that include a retrieval component of a surgical halo extending through a body wall.

In the scenario 1200, a cannula 1204 includes a surgical halo 1206 (e.g., the cannula 802 of FIG. 8). In this scenario, the cannula 1204 was inserted into a body wall 1208 while the surgical halo 1206 was attached to the cannula 1204, such as described above with respect to FIG. 11. The surgical halo 1206 includes a retrieval component 1210. In this scenario, a portion of the retrieval component 1210 is attached to the surgical halo 1206, a portion of retrieval component 1210 extends upward and passes through the body wall 1208, and a portion of the retrieval component 1210 is located outside of the body. For instance, portions of the retrieval component 1210 are located between an exterior surface of a cannula rod of the cannula and an edge of the body wall 1208 (e.g., an edge corresponding to an incision through the body wall 1208).

In the scenario 1202, a cannula 1212 includes a surgical halo 1214 (e.g., the cannula 1004 and the surgical halo 1006c of FIG. 10). In this scenario, the cannula 1212 was inserted into a body wall 1216 and the surgical halo 1214 was subsequently attached to the cannula 1212, such as described above with respect to FIG. 10. The surgical halo 1214 includes a retrieval component 1218. In this scenario, a portion of the retrieval component 1218 is attached to the surgical halo 1214, a portion of retrieval component 1218 travels through the body cavity to a cannula 1220, a portion of the retrieval component 1218 passes through the cannula 1220, and a portion of the retrieval component 1218 is located outside of the body. For instance, the surgical halo 1214 was inserted into the body cavity by passing the surgical halo 1214 through the cannula 1220. During insertion into the body cavity, a portion of the retrieval component 1218 was held outside of the body (e.g., held by a human user, attached to another object outside of the body, and so forth).

In this way, a portion of the retrieval component 1210 and a portion of the retrieval component 1218 remain outside of the body even while the respective surgical halos are attached to portions of cannulas inside of the body. This enables the ability to easily retrieve the surgical halos 1206 or 1214 in the event that they become detached from their respective cannulas while inside of the body, such as by pulling on the retrieval components 1210 or 1218 to pull their respective surgical halos out of the body.

Functionality, features, and concepts described in relation to different figures and examples in this document may be interchanged among one another and are not limited to implementation in the context of a particular figure or procedure. Moreover, scenarios associated with different representative procedures and corresponding figures herein may be applied together and/or combined in different ways. Thus, individual functionality, features, and concepts described in relation to different example devices, scenarios, and procedures herein may be used in any suitable combinations and are not limited to the particular combinations represented by the enumerated examples in this description.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A device comprising:
an elastically deformable body including a canopy and a tube;
the tube, while in an undeformed configuration, extending along a longitudinal axis, the tube having an interior surface with a radius perpendicular to the longitudinal axis, and an exterior surface;
the canopy, while in the undeformed configuration, having:
a lower surface forming a concave curve extending from a first portion of the exterior surface to an outer edge; and
an upper surface forming a convex curve extending from a second portion of the exterior surface to the outer edge, configured with a portion of the upper surface further from the longitudinal axis than a corresponding portion of the lower surface; and
the canopy, while in a deformed configuration, configured with the portion of the lower surface further from the longitudinal axis than the portion of the upper surface.

2. The device of claim 1, wherein the elastically deformable body further includes a marker component, and the marker component includes a material visible outside of the visible spectrum of light.

3. The device of claim 2, wherein the material is a radiopaque marker.

4. The device of claim 3, wherein the radiopaque marker is a radiopaque substance and the elastically deformable body is formed of a material that includes the radiopaque substance.

5. The device of claim 1, further comprising a retrieval component coupled with the elastically deformable body.

6. The device of claim 5, wherein the retrieval component includes a radiopaque marker.

7. The device of claim 5, wherein:
the elastically deformable body further includes at a channel between the upper surface and the lower surface; a
a first portion of the retrieval component is disposed within the channel; and
a second portion of the retrieval component is disposed around at least part of the tube.

8. The device of claim 1, wherein the tube has a first thickness proximal to the canopy and a second thickness proximal to an end of the tube, the first thickness greater than the second thickness.

9. The device of claim 1, wherein the canopy has a first thickness proximal to the tube and a second thickness proximal to the outer edge, the first thickness greater than the second thickness.

10. The device of claim 1, wherein the deformed configuration is configured with at least a portion of the lower surface generally parallel to the longitudinal axis.

11. The device of claim 1, wherein the radius in a second deformed configuration is greater than the radius in the undeformed configuration.

12. The device of claim 1, wherein the radius in a second deformed configuration is greater than the radius in the undeformed configuration, and the canopy, while in the second deformed configuration, is configured with the lower surface forming the concave curve and the upper surface forming the convex curve.

13. The device of claim 1, further comprising a cannula with a body extending along the longitudinal axis, an inner surface with an inner radius perpendicular to the longitudinal axis, and an outer surface generally parallel with the inner surface, wherein the interior surface of the elastically deformable body is coupled to the outer surface of the cannula.

14. A method comprising:
deforming an elastically deformable surgical halo with a body including a canopy and a tube by applying a force against the surgical halo to configure the surgical halo into a deformed state, the surgical halo in an undeformed state including:
the tube extending along a longitudinal axis, the tube having an interior surface with a radius perpendicular to the longitudinal axis, and an exterior surface; and
the canopy having a lower surface forming a concave curve extending from a first portion of the exterior surface to an outer edge and an upper surface forming a convex curve extending from a second portion of the exterior surface to the outer edge;
moving the surgical halo in the deformed state along an outer surface of a cannula, the outer surface having dimensions greater than the radius in the undeformed state; and
affixing the surgical halo to the outer surface of the cannula by releasing the force against the surgical halo.

15. The method of claim 14, further comprising, prior to the deforming:
deforming the surgical halo by applying a second force against the surgical halo to configure the surgical halo into a second deformed state;
moving the surgical halo in the second deformed state through an interior radius of a second cannula; and
returning the surgical halo to the undeformed state by releasing the second force against the surgical halo.

16. The method of claim 14, further comprising, subsequent to the affixing, moving the surgical halo through a body wall.

17. The method of claim 16, wherein in the undeformed state a portion of the upper surface is further from the longitudinal axis than a corresponding portion of the lower surface; and
the moving includes:
deforming the surgical halo to configure the surgical halo into a second deformed state, the surgical halo in the second deformed state the portion of the lower surface is further from the longitudinal axis than the portion of the upper surface;
passing the surgical halo through the body wall while the surgical halo is in the second deformed state; and
returning the surgical halo to the undeformed state.

18. A cannula comprising:
a surgical port head;
a cannula shaft connected to the surgical port head, the cannula shaft extending along a longitudinal axis and having an inner surface with a radius perpendicular to the longitudinal axis, and an outer surface; and
an elastically deformable surgical halo affixed to the outer surface of the cannula, the surgical halo including a body with a canopy and a tube, the surgical halo in an undeformed configuration including:
the tube extending along the longitudinal axis, the tube having an interior surface with a radius perpendicular to the longitudinal axis, and an exterior surface; and
the canopy having a lower surface forming a concave curve extending from a first portion of the exterior surface to an outer edge and an upper surface forming a convex curve extending from a second portion of the exterior surface to the outer edge.

19. The cannula of claim 18, wherein the outer surface of the cannula includes a groove with a radius perpendicular to the longitudinal axis, and wherein a portion of the interior surface of the surgical halo is deformed inward at a location corresponding to the groove.

20. The cannula of claim 18, wherein the exterior surface of the cannula has dimensions greater than the radius of the interior surface of the surgical halo in the undeformed configuration, and wherein the surgical halo exerts an inward compressive force toward the longitudinal axis while the surgical halo is in a deformed state with the radius being greater than the radius in the undeformed configuration.
